(19) United States
(12) Patent Application Publication
(10) Pub. No.: US 2009/0264485 A1
(43) Pub. Date: Oct. 22, 2009

(54) Pyrazole Derivatives, Their Manufacture and Their Use as Pharmaceutical Agents

(76) Inventors: Hans-Willi Krell, Penzberg (DE); Joerg Middeldorff, Bernried (DE); Ulrike Reiff, Penzberg (DE); Thomas von Hirschheydt, Penzberg (DE); Edgar Voss, Bichl (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/989,218

(22) PCT Filed: Aug. 7, 2006
(86) PCT No.: PCT/EP2006/007789

§ 371 Date: Jan. 22, 2008

(30) Foreign Application Priority Data
Aug. 8, 2005 (EP) 05017192.5

(51) Int. Cl. A61K 31/422; C07D 413/12; A61P 35/00

(52) U.S. Cl. 514/374; 548/235

(57) ABSTRACT

Objects of the present invention are the compounds of formula I

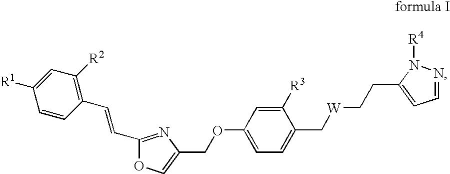

their pharmaceutically acceptable salts, enantiomeric forms, diastereoisomers and racemates, the preparation of the above-mentioned compounds, pharmaceutical compositions containing them and their manufacture, as well as the use of the above-mentioned compounds in the control or prevention of illnesses such as cancer.

The present invention relates to novel pyrazole derivatives, to a process for their manufacture, pharmaceutical compositions containing them and their manufacture as well as the use of these compounds as pharmaceutically active agents.

### BACKGROUND OF THE INVENTION

Protein tyrosine kinases (PTKs) catalyze the phosphorylation of tyrosyl residues in various proteins involved in the regulation of cell growth and differentiation (Wilks et al., Progress in Growth Factor Research 97 (1990)₂; Chan, A. C., and Shaw, A. S., Curr. Opin. Immunol. 8 (1996) 394-401). Such PTKs can be divided into receptor tyrosine kinases (e.g. EGFR/HER-1, c-erB2/HER-2, c-met, PDGFr, FGFr) and non-receptor tyrosine kinases (e.g. src, lck). It is known that many oncogenes encode proteins which are aberrant tyrosine kinases capable of causing cell transformation (Yarden, Y., and Ullrich, A., Annu. Rev. Biochem. 57 (1988) 443-478; Larsen et al., Ann. Reports in Med. Chem., 1989, Chpt. 13). Also over-expression of a normal proto-oncogenic tyrosine kinase may result in proliferative disorders.

It is known that receptor tyrosine kinases of the HER-family like HER-2 and EGFR (HER-1) are frequently aberrantly expressed in common human cancers such as breast cancer, gastrointestinal cancer (colon, rectal or stomach cancer), leukemia and ovarian, bronchial and pancreatic cancer. High levels of these receptors correlate with poor prognosis and response to treatment (Wright, C., et al., Br. J. Cancer 65 (1992) 118-121).

Accordingly, it has been recognized that inhibitors of receptor tyrosine kinases are useful as selective inhibitors of the growth of mammalian cancer cells. Therefore several small molecule compounds as well as monoclonal antibodies are in clinical trials for the treatment of various types of cancer (Baselga, J., and Hammond, L. A., Oncology 63 (Suppl. 1) (2002) 6-16; Ranson, M., and Sliwkowski, M. X., Oncology 63 (suppl. 1) (2002) 17-24).

Some substituted oxazoles are known in the art. WO 03/059907 relates to nitrogenous heterocycles useful as anticancer agents. WO 98/03505, EP 1 270 571, WO 01/77107 and WO 03/031442 disclose heterocyclic compounds as tyrosine kinase inhibitors.

However there remains a need for new compounds with improved therapeutic properties, such as enhanced activity, decreased toxicity, better solubility and improved pharmacokinetic profile, to name only a few.

### SUMMARY OF THE INVENTION

The present invention relates to compounds of the general formula I,

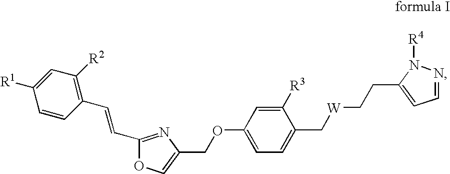

wherein
R¹ is halogenated alkyl, halogenated alkoxy or halogen;
R² is hydrogen or halogen;
R³ is hydrogen or alkyl;
R⁴ is alkyl;
W is —O—, —S—, —S(O)— or —S(O)₂—;
and all pharmaceutically acceptable salts thereof.

The compounds of the present invention show activity as inhibitors of the HER-signalling pathway and therefore possess anti-proliferative activity. Objects of the present invention are the compounds of formula I and their pharmaceutically acceptable salts, enantiomeric forms, diastereoisomers and racemates, the preparation of the above-mentioned compounds, pharmaceutical compositions containing them and their manufacture as well as the use of the above-mentioned compounds in the control or prevention of illnesses, especially of illnesses and disorders as mentioned above like common human cancers (e.g. breast cancer, gastrointestinal cancer (colon, rectal or stomach cancer), leukaemia and ovarian, bronchial and pancreatic cancer) or in the manufacture of corresponding pharmaceutical compositions.

### DETAILED DESCRIPTION OF THE INVENTION

As used herein, the term “alkyl” means a saturated, straight-chain or branched-chain hydrocarbon containing from 1 to 5 carbon atoms, preferably from 1 to 3 carbon atoms, such as methyl, ethyl, n-propyl, isopropyl, n-butyl, 2-butyl, t-butyl, n-pentyl, 3-methyl-butyl or 2-methyl-butyl.

In a preferred embodiment, the term “alkyl” as used in R³ denotes a (C₁-C₂)alkyl, preferably methyl and the term “alkyl” as used in R⁴ denotes a(C₁-C₂)alkyl, preferably methyl.

As used herein, the term “halogenated alkyl” means an alkyl as defined above which is substituted with one or several halogen atoms, preferably fluorine or chlorine, especially fluorine. Examples are trifluoromethyl, 2,2,2-trifluoroethyl, perfluoroethyl and the like, preferably trifluoromethyl.

The term “halogenated alkoxy” as used herein means an alkoxy group as defined above which is substituted one or several times by halogen, preferably by fluorine or chlorine, especially by fluorine. Examples are difluoromethoxy, trifluoromethoxy, 2,2,2-trifluoroethoxy, perfluoroethoxy and the like, preferably trifluoromethoxy and difluoromethoxy and especially trifluoromethoxy.

The term “halogen” as used herein means fluorine, chlorine and bromine, preferably fluorine or chlorine.

In a preferred embodiment, the term “halogen” as used in R¹ denotes fluorine or chlorine, preferably chlorine and the term “halogen” as used in R² denotes fluorine or chlorine, preferably fluorine.

As used herein, when referring to the receptor tyrosine kinases of the HER-family like HER-2 and EGFR (HER-1), the acronym “HER” refers to human epidermal receptor and the acronym “EGFR” refers to epidermal growth factor receptor.

As used herein, in relation to mass spectrometry (MS) the term “ES+” refers to positive electrospray ionization mode.

As used herein, the term “a therapeutically effective amount” of a compound means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration, it may be given as continuous infusion.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

An embodiment of the invention are the compounds of formula I, wherein
R² is hydrogen.

Another embodiment of the invention are the compounds of formula I, wherein
R¹ is halogenated alkoxy.

Another embodiment of the invention are the compounds of formula I, wherein
R¹ is halogenated alkoxy; and
R² is hydrogen.

Another embodiment of the invention are the compounds of formula I, wherein
R¹ is halogenated alkoxy;
R² is hydrogen; and
R³ is alkyl.

Another embodiment of the invention are the compounds of formula I, wherein
R¹ is halogenated alkoxy; and
R² is hydrogen; and
W is —O—.

Such a compound is for example:
4-{3-Methyl-4-[2-(2-methyl-2H-pyrazol-3-yl)-ethoxymethyl]-phenoxymethyl}-2-[(E)-2-(4-trifluoromethoxy-phenyl)-vinyl]-oxazole.

Another embodiment of the invention are the compounds of formula I, wherein
R¹ is halogenated alkoxy;
R² is hydrogen; and
W is —S(O)—.

Such compounds, for example, may be selected from the group consisting of:
4-{3-Methyl-4-[2-(2-methyl-2H-pyrazol-3-yl)-ethanesulfinylmethyl]-phenoxymethyl}-2-[(E)-2-(4-trifluoromethoxy-phenyl)-vinyl]-oxazole; and
4-{4-[2-(2-Methyl-2H-pyrazol-3-yl)-ethanesulfinylmethyl]-phenoxymethyl}-2-[(E)-2-(4-trifluoromethoxy-phenyl)-vinyl]-oxazole.

Another embodiment of the invention are the compounds of formula I, wherein
R¹ is halogenated alkyl.

Another embodiment of the invention are the compounds of formula I, wherein
R¹ is halogenated alkyl; and
W is —O—.

Such compounds, for example, may be selected from the group consisting of:
4-{3-Methyl-4-[2-(2-methyl-2H-pyrazol-3-yl)-ethoxymethyl]-phenoxymethyl}-2-[(E)-2-(4-trifluoromethyl-phenyl)-vinyl]-oxazole; and
2-[(E)-2-(2-Fluoro-4-trifluoromethyl-phenyl)-vinyl]-4-{3-methyl-4-[2-(2-methyl-2H-pyrazol-3-yl)-ethoxymethyl]-phenoxymethyl}-oxazole.

Another embodiment of the invention are the compounds of formula I, wherein
R¹ is halogenated alkyl; and
R² is hydrogen.

Another embodiment of the invention are the compounds of formula I, wherein
R¹ is halogenated alkyl; and
R² is hydrogen; and
W is —O—.

Another embodiment of the invention are the compounds of formula I, wherein
R¹ is halogenated alkyl; and
R² is fluorine.

Another embodiment of the invention are the compounds of formula I, wherein
R¹ is halogenated alkyl;
R² is fluorine; and
W is —O—.

Another embodiment of the invention are the compounds of formula I, wherein
R¹ is halogenated alkyl;
R² is hydrogen; and
W is —S(O)—.

Such compounds, for example, may be selected from the group consisting of:
4-{3-Methyl-4-[2-(2-methyl-2H-pyrazol-3-yl)-ethanesulfinylmethyl]-phenoxymethyl}-2-[(E)-2-(4-trifluoromethyl-phenyl)-vinyl]-oxazole; and
4-{4-[2-(2-Methyl-2H-pyrazol-3-yl)-ethanesulfinylmethyl]-phenoxymethyl}-2-[(E)-2-(4-trifluoromethyl-phenyl)-vinyl]-oxazole.

Another embodiment of the invention are the compounds of formula I, wherein
R¹ is halogen.

Another embodiment of the invention are the compounds of formula I, wherein
R¹ is halogen; and
R² is hydrogen.

Another embodiment of the invention are the compounds of formula I, wherein
R¹ is halogen;
R² is hydrogen; and
W is —O—.

Such a compound is for example:
2-[(E)-2-(4-Chloro-phenyl)-vinyl]-4-{3-methyl-4-[2-(2-methyl-2H-pyrazol-3-yl)-ethoxymethyl]-phenoxymethyl}-oxazole.

Another embodiment of the invention are the compounds of formula I, wherein
R¹ is halogen;
R² is hydrogen; and
W is —S(O)—.

Such a compound is for example:
2-[(E)-2-(4-Chloro-phenyl)-vinyl]-4-{3-methyl-4-[2-(2-methyl-2H-pyrazol-3-yl)-ethanesulfinylmethyl]-phenoxymethyl}-oxazole.

Another embodiment of the invention are the compounds of formula I, wherein
R³ is alkyl.

An embodiment of the invention are the compounds of formula I, wherein
R² is hydrogen; and
R³ is alkyl.

Another embodiment of the invention are the compounds of formula I, wherein
R³ is hydrogen.

An embodiment of the invention are the compounds of formula I, wherein
R² is hydrogen; and
R³ is hydrogen.

Another embodiment of the invention are the compounds of formula I, wherein
W is —O—.

Another embodiment of the invention are the compounds of formula I, wherein
W is —S—.

Another embodiment of the invention are the compounds of formula I, wherein
W is —S(O)—.

Another embodiment of the invention are the compounds of formula I, wherein
W is —S(O)₂—

Another embodiment of the invention are the compounds of formula I, wherein
R¹ is halogenated alkyl; and
R² is hydrogen.

Another embodiment of the invention are the compounds of formula I, wherein
R¹ is halogenated alkyl;
R² is hydrogen; and
W is —S(O)₂—

Another embodiment of the invention are the compounds of formula I, wherein
W is —O—, —S(O)— or —S(O)₂—.

Another embodiment of the invention are the compounds of formula I, wherein
R² is hydrogen; and
W is —O—, —S(O)— or —S(O)₂—.

Another embodiment of the invention are the compounds of formula I, wherein
R¹ is halogenated alkoxy;
R² is hydrogen; and
W is —O—, —S(O)— or —S(O)₂—.

Another embodiment of the invention are the compounds of formula I, wherein
W is —O— or —S(O)—.

Another embodiment of the invention are the compounds of formula I, wherein
R² is hydrogen; and
W is —O— or —S(O)—.

Another embodiment of the invention are the compounds of formula I, wherein
R¹ is halogenated alkoxy;
R² is hydrogen; and
W is —O— or —S(O)—.

Another embodiment of the invention are the compounds of formula I, wherein
R³ is alkyl; and
W is —O— or —S(O)—.

Another embodiment of the invention are the compounds of formula I, wherein
R² is hydrogen or fluorine.

Another embodiment of the invention are the compounds of formula I, wherein
R² is hydrogen or fluorine; and
W is —O—, —S(O)— or —S(O)₂—.

Another embodiment of the invention are the compounds of formula I, wherein
R¹ is halogenated alkoxy;
R² is hydrogen or fluorine; and
W is —O—, —S(O)— or —S(O)₂—.

Another embodiment of the invention are the compounds of formula I, wherein
R¹ is halogenated alkyl;
R² is hydrogen or fluorine; and
W is —O—, —S(O)— or —S(O)₂—.

Another embodiment of the invention are the compounds of formula I, wherein
R¹ is halogen;
R² is hydrogen or fluorine; and
W is —O—, —S(O)— or —S(O)₂—.

Another embodiment of the invention are the compounds of formula I, wherein
R¹ is halogenated alkyl, halogenated alkoxy or chlorine;
R² is hydrogen or fluorine;
R³ is alkyl; and
W is —O— or —S(O)—.

Another embodiment of the invention are the compounds of formula I, wherein
R¹ is halogenated alkoxy;
R² is hydrogen;
R³ is alkyl; and
W is —O— or —S(O)—.

Another embodiment of the invention are the compounds of formula I, wherein
R¹ is halogenated alkyl, halogenated alkoxy or chlorine;
R² is hydrogen or fluorine;
R³ is alkyl; and
W is —O—.

Another embodiment of the invention are the compounds of formula I, wherein
R¹ is halogenated alkoxy;
R² is hydrogen;
R³ is alkyl; and
W is —O—.

Another embodiment of the invention are the compounds of formula I, wherein
R¹ is halogenated alkyl, halogenated alkoxy or chlorine;
R² is hydrogen;
R³ is hydrogen or alkyl; and
W is —S(O)—.

Another embodiment of the invention are the compounds of formula I, wherein
R¹ is halogenated alkyl, halogenated alkoxy or chlorine;
R² is hydrogen;
R³ is alkyl; and
W is —S(O)—.

Another embodiment of the invention are the compounds of formula I, wherein
R¹ is halogenated alkoxy;
R² is hydrogen;
R³ is hydrogen or alkyl; and
W is —S(O)—.

Another embodiment of the invention is a process for the manufacture of the compounds of formula I, wherein
(a) the compound of formula V

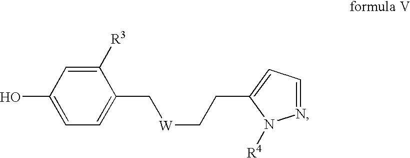

wherein R³, and R⁴ have the significance as given in formula I above and
W is —O—, —S— or —S(O)—,
is reacted with a compound of formula IV

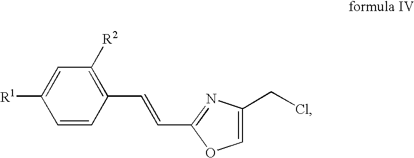

wherein R¹ and R² have the significance given in formula I above to give the respective compound of formula I, wherein W is —O—, —S— or —S(O)—;
(b) if desired, said compound of formula I, wherein W is —S— or —S(O)— is oxidized to give the respective compound of formula I, wherein W is —S(O)₂—;
(c) said compound is isolated from the reaction mixture, and
(d) if desired, converted into a pharmaceutically acceptable salt.

The compounds of formula I, or a pharmaceutically acceptable salt thereof, which are subject of the present invention, may be prepared by any process known to be applicable to the preparation of chemically-related compounds. Such processes, when used to prepare a compound of the formula I, or a pharmaceutically-acceptable salt thereof, are illustrated by the following representative schemes 1 to 4 and examples in which, unless otherwise stated, R¹, R², R³, R⁴, and W has the significance given herein before. Necessary starting materials are either commercially available or they may be obtained by standard procedures of organic chemistry. The preparation of such starting materials is e.g. described within the accompanying examples or in schemes 1 to 4. Alternatively necessary starting materials are obtainable by analogous procedures to those illustrated which are within the ordinary skill of an organic chemist.

### Scheme 1

A preferred method for the synthesis of the compounds of formula I is described in scheme 1.

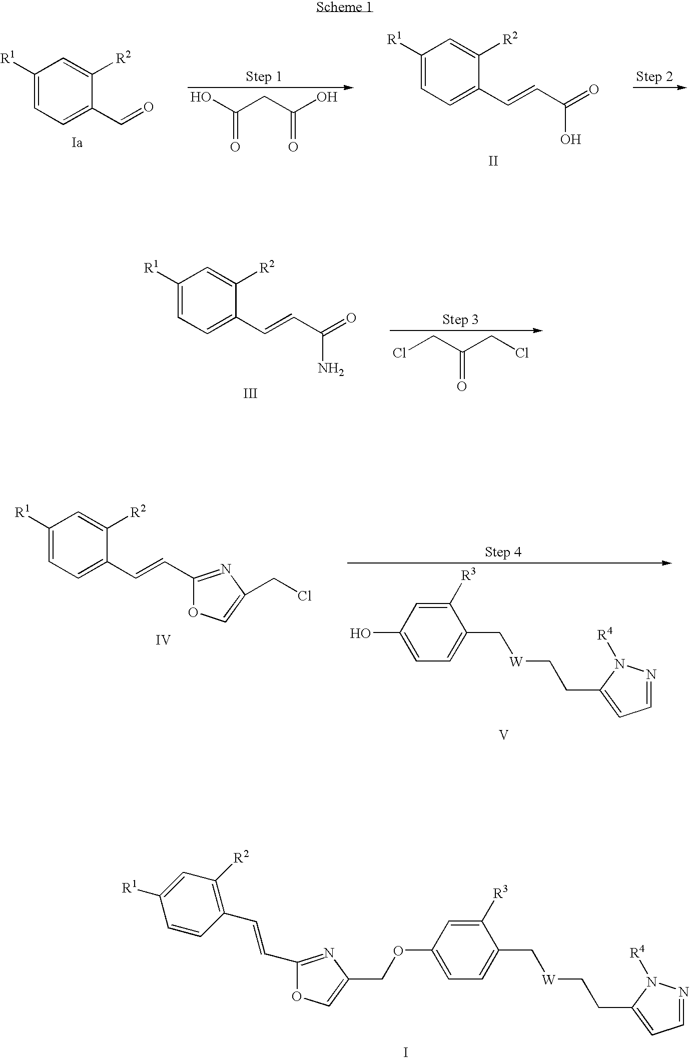

In scheme 1, R¹, R², R³, R⁴ and W have the significance as given above for formula I.

Step 1, scheme 1 of the reaction sequence is a Knoevenagel condensation of the benzaldehydes of formula Ia with malonic acid and concomitant decarboxylation, yielding acrylic acids of formula II. The reaction is typically carried out in solvents like pyridine, N-methylpyrrolidinone, acetonitrile, N,N-dimethylformamide and mixtures thereof at temperatures up to 140° C. Typically used bases are piperidine, triethylamine and diisopropylamine.

In Step 2, scheme 1, the obtained acrylic acids of formula II are converted into their corresponding amides of formula III by standard methods for someone skilled in the art, e.g. by activating the carboxylic group in formula II with oxalyl chloride in solvents like tetrahydrofuran, dichloromethane, N,N-dimethylformamide and mixtures thereof at temperatures varying from −30° C. to 40° C. The addition of ammonia yields said amides of formula III.

In Step 3, scheme 1, the chlorides of formula IV can be synthesized by a commonly known method or a modification thereof. Amides of formula III and 1,3-dichloroacetone are subjected to a condensation/dehydration sequence yielding the compounds of formula IV. Typical solvents for reactions of this kind are toluene, xylene, benzene, acetone and chloroform. If desired, the reaction can be carried out under solvent free conditions. The reaction temperatures may vary from 50° C. to 150° C.

In Step 4, scheme 1, the derivatives of formula I can be obtained e.g. by alkylation of compounds of formula V with compounds of formula IV. Typically the alkylation is carried out in solvents like N,N-dimethylformamide (DMF), methanol, ethanol and isopropanol sometimes in the presence of bases such as sodium methylate, sodium hydride or lithium diisopropyl amide and the like. The reaction temperatures may vary from 0° C. to 150° C. Sometimes potassium iodide or sodium iodide is added to the reaction mixture.

### Scheme 2

The phenolic intermediates of formula V may be prepared in a two step reaction as shown in scheme 2.

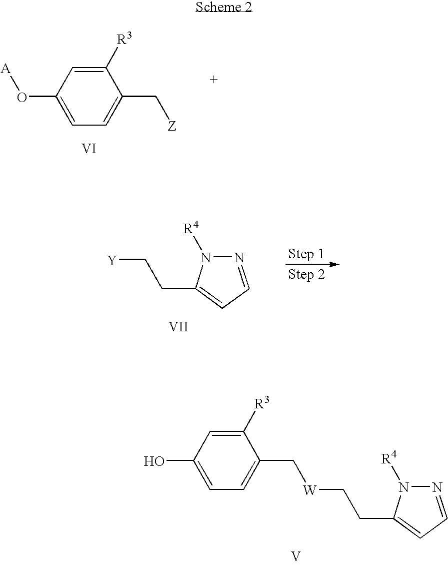

In scheme 2, R³, R⁴ and W have the significance as given above for formula I and A is a hydroxy protecting group such as propen-3-yl (allyl), triphenylmethyl (trityl) and silyl groups (e.g. tert.-butyl-dimethyl-silyl, triisopropyl-silyl) and the like.

Step 1, scheme 2 is an alkylation reaction of a compound of formula VI with a compound of formula VII. Depending on the nature of W different variations of this reaction are known and the significance of Z and Y may vary accordingly:

In the case that W is —O—,

A denotes a suitable protecting group as defined below,
one of Z and Y denotes a hydroxy group,
while the other denotes a suitable leaving group LG as defined below
   and in the case that W is —S—,
   A denotes a suitable protecting group as defined below,
   Z denotes a thiol group and
   Y denotes a suitable leaving group LG as defined below.

Reactions of compounds of formula VI with compounds of formula VII are well known in the art. Typically, such alkylation reaction may be carried out in solvents like N,N-dimethylformamide (DMF), methanol, ethanol and isopropanol. Typical bases for this reaction are alkaline carbonates, sodium methylate, sodium hydride or lithium diisopropyl amide. The reaction temperatures may vary from 20° C. to 150° C. Other preferred alkylation procedures make use of alkaline carbonates as bases in solvents like ketones, for example cesium carbonate in butanone at reflux temperature, or sodium hydride in DMF at room temperature. Suitable leaving groups LG are those typically used in alkylation reactions and well known to the skilled artisan. Examples of such leaving groups LG are, among others, the anions of halogens, especially iodide, bromide or chloride, p-toluenesulfonate (tosylate), methanesulfonate (mesylate), trifluoromethansulfonate (triflate) or the azido group.

To obtain the compounds wherein W is —S(O)— or —S(O)₂—, the intermediate thioethers (W is —S—) can be oxidized with e.g. meta-chloro-perbenzoic acid (mCPBA) or Oxone® to yield the corresponding sulfoxides or sulfones (W is —S(O)— or —S(O)₂—).

In Step 2, scheme 2 the subsequent removal of the protecting group A is performed. The hydroxy protecting group A as mentioned herein is a conventional protecting group as known by the skilled artisan. Examples are propen-3-yl (allyl), triphenylmethyl (trityl) and silyl groups, e.g. tert.-butyl-dimethyl-silyl, triisopropyl-silyl.

Removal of a protecting group on a hetero atom depends on the nature of such group. Typical examples are the removal of a trityl group under acidic conditions, for example with aqueous formic acid in tetrahydrofuran (THF) under reflux or the removal of a tert-butoxycarbonyl group with trifluoroacetic acid in dichloromethane at room temperature or the removal of a substituted silyl group with tetrabutylammonium fluoride in aqueous THF at room temperature. An allyl group can smoothly be removed by treating the substrate with catalytic amounts of a palladium complex, e.g. Pd(PPh₃)₄ in dichloromethane in presence of an allyl-acceptor such as 1,3-dimethylbarbituric acid.

Compounds of formula V are new and also subject of this invention.

### Scheme 3

Y in formula VII above is a hydroxy group or a leaving group LG, and the compounds of the formula VII are prepared as outlined in scheme 3. These are named accordingly VII-a (for Y is hydroxy) and VII-b (for Y is a leaving group LG).

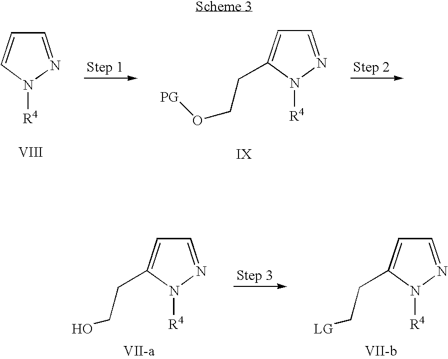

In scheme 3, R⁴ has the significance as given above for formula I, LG is a leaving group as defined above, e.g. iodide, bromide or chloride, p-toluenesulfonate (tosylate), methanesulfonate (mesylate), trifluoromethansulfonate (triflate) or the azido group, and PG is a hydroxy protecting group such as a silyl group (e.g. ter.-butyl-dimethyl-silyl, triisopropyl-silyl), an acetal group (e.g. 2-tetrahydro-pyran) or other suitable protecting groups.

In Step 1, scheme 3 the appropriately N-substituted pyrazoles VIII are converted to the compounds of formula IX by regioselective deprotonation with n-buthyl lithium and subsequent reaction with O-protected 2-haloethanols (e.g. (2-bromo-ethoxy)-tert-butyl-dimethyl-silane or 2-(2-bromo-ethoxy)-tetrahydro-pyran) in inert solvents like tetrahydrofurane or diethylether. Temperatures may vary from −40° C. to −80° C. for the lithiation step and warming up to room temperature after addition of all reagents.

With Step 2, scheme 3 the hydroxyl group is deprotected using commonly known methods providing 5-(2-hydroxyethyl)pyrazoles of formula VII-a. Removal of a protecting group on a hetero atom depends on the nature of such group. Typical examples are the removal of a silyl group using a fluoride source e.g. NaF/HBr (in methanol/water) or tetrabutylammoniumfluoride at room temperature or the cleavage of an acetal under acidic conditions.

In Step 3, scheme 3 the hydroxyl group is converted to a leaving group “LG” yielding the compounds of formula VII-b. Suitable leaving groups are those typically used in alkylation reactions and well known to the skilled artisan. Examples of such leaving groups are, among others, the anions of halogens, especially iodode, bromide or chloride, p-toluensulfonate (tosylate), methanesulfonate (mesylate), trifluoromethansulfonate (triflate) or the azido group. Such leaving groups can be introduced by standard procedures of organic chemistry e.g. by reaction of the alcohols of formula VII-a with trifluoromethanesulfonic acid anhydride yielding the corresponding triflates, with p-toluenesulfonyl chloride yielding the corresponding tosylate or with inorganic acid halides e.g. SOCl₂, PCl₃, PCl₅ or PBr₃ in the presence of a base e.g. pyridine, lutidine or triethylamine yielding the corresponding halides, respectively. The iodides can be synthesized by exchange of a leaving group as denoted above with iodide by means of a finkelstein reaction wich is well known by one of ordinary skill in the art.

Compounds of formula VII are new and also subject of this invention.

### Scheme 4

Z in formula VI above is a hydroxy group, a leaving group LG or thiol group, and the compounds of the formula VI are prepared as outlined in scheme 4. These compounds are named accordingly VI-a (Z is a hydroxy group), VI-b (Z is a leaving group LG) and VI-c (Z is a thiol group).

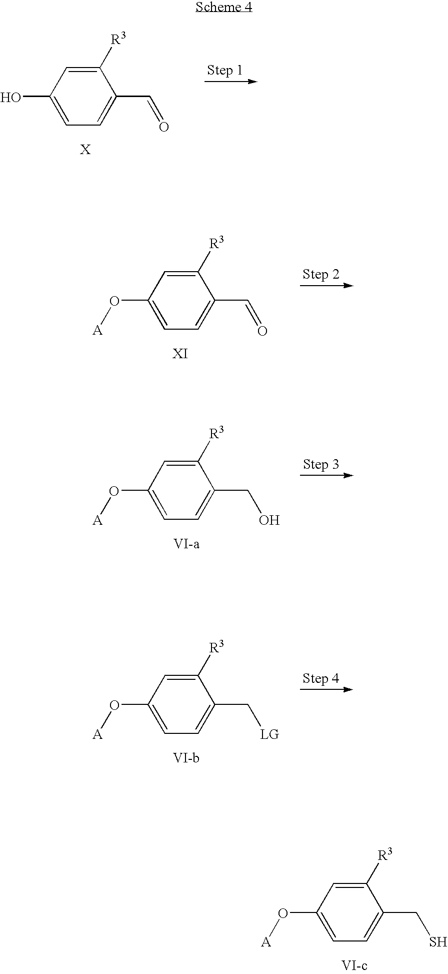

In scheme 4, R³ has the significance as given above for formula I, A is a hydroxy protecting group such as propen-3-yl (allyl), triphenylmethyl (trityl) and silyl groups (e.g. tert.-butyl-dimethyl-silyl, triisopropyl-silyl) and the like and LG is a leaving group as defined above, e.g. iodide, bromide or chloride, p-toluenesulfonate (tosylate), methanesulfonate (mesylate), trifluoromethansulfonate (triflate) or the azido group.

In step 1, scheme 4 a the hydroxy protecting group A is introduced to the phenolic hydroxyl group by standard methods well known to the skilled artisan e.g. reaction of compounds of formula X with allylbromide or triphenylmethyl chloride in the presence of a base e.g. potassium carbonate yielding the corresponding ethers or a silyl chloride e.g. ter.-butyl-dimethylsilyl chloride in the presence of a base like e.g. pyridine, lutidine or imidazole yielding the corresponding silyl ethers.

In step 2, scheme 4 the carbonyl group of the compounds of formula IX is reduced by complex inorganic hydrides e.g. among others lithium aluminium hydride or dibutyl aluminium hydride yielding the benzylic alcohols VI-a.

In step 3, scheme 4 the hydroxyl group of the compounds of formula VI-a is converted to a leaving group in the same way as described for step 3, scheme 3 yielding the compounds of formula VI-b.

In step 4, scheme 4 compounds of the formula VI-b are converted to the corresponding thiols by reacting with thiourea and subsequent cleavage of the intermediate thiouronium chlorides under basic conditions.

For protecting and deprotecting of a hydroxyl group in Schemes 1 to 4, many more methods than outlined above may be suitable. A comprehensive overview is given in T. W. Greene and P. G. M. Wuts, Protective Groups in Organic Synthesis, 3^{rd} edition (1999) Whiley Interscience New York, Chichester, Weinheim, Brisbane, Toronto, Singapore.

The compounds of formula I can contain one or several chiral centers and can then be present in a racemic or in an optically active form. The racemates can be separated according to known methods into the enantiomers. For instance, diastereomeric salts which can be separated by crystallization are formed from the racemic mixtures by reaction with an optically active acid such as e.g. D- or L-camphorsulfonic acid. Alternatively separation of the enantiomers can also be achieved by using chromatography on chiral HPLC-phases which are commercially available.

The compounds according to the present invention may exist in the form of their pharmaceutically acceptable salts. The term “pharmaceutically acceptable salt” refers to conventional acid-addition salts that retain the biological effectiveness and properties of the compounds of formula I and are formed from suitable non-toxic organic or inorganic acids. Examples of acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, naphthalenesulfonic acid, naphthalenedisulfonic acid, methanesulfonic acid, ethanesulfonic acid and the like. The chemical modification of a pharmaceutical compound (i.e. a drug) into a salt is a technique well known to pharmaceutical chemists to obtain improved physical and chemical stability, hygroscopicity, flowability and solubility of compounds. See, e.g., Stahl, P. H., and Wermuth, G., (editors), Handbook of Pharmaceutical Salts, Verlag Helvetica Chimica Acta (VHCA), Zürich, (2002) or Bastin, R. J., et al., Organic Proc. Res. Dev. 4 (2000) 427-435.

Preferred are the pharmaceutically acceptable salts, which are formed with p-toluenesulfonic acid, naphthalenesulfonic acid, naphthalenedisulfonic acid, methanesulfonic acid and hydrochloric acid.

The compounds of formula I can contain one or several chiral centers and can then be present in a racemic or in an optically active form. The racemates can be separated according to known methods into the enantiomers. For instance, diastereomeric salts which can be separated by crystallization are formed from the racemic mixtures by reaction with an optically active acid such as e.g. D- or L-camphorsulfonic acid. Alternatively separation of the enantiomers can also be achieved by using chromatography on chiral HPLC-phases which are commercially available.

Medicaments or pharmaceutical compositions containing a compound of the present invention or a pharmaceutically acceptable salt thereof and a therapeutically acceptable carrier are also an object of the present invention, as is a process for their production, which comprises bringing one or more compounds of the present invention and/or pharmaceutically acceptable salts and, if desired, one or more other therapeutically valuable substances into a galenical administration form together with one or more therapeutically acceptable carriers.

In accordance with the invention the compounds of the present invention as well as their pharmaceutically acceptable salts are useful in the control or prevention of illnesses. Based on their HER-signalling pathway inhibition and their antiproliferative activity, said compounds are useful for the treatment of diseases such as cancer in humans or animals and for the production of corresponding pharmaceutical compositions. The dosage depends on various factors such as manner of administration, species, age and/or individual state of health.

Another embodiment of the invention is pharmaceutical composition, containing one or more compounds of formula I together with pharmaceutically acceptable carriers.

Still another embodiment of the invention is said pharmaceutical composition for the inhibition of tumor growth.

Still another embodiment of the invention is the use of a compound of formula I for the inhibition of tumor growth.

Still another embodiment of the invention is the use of a compound of formula I for the treatment of cancer.

Still another embodiment of the invention is the use of a compound of formula I for the manufacture of corresponding pharmaceutical compositions for the inhibition of tumor growth.

Another embodiment of the invention is a pharmaceutical composition comprising a therapeutically effective amount of a compound according to formula I as active ingredients and a pharmaceutically acceptable carrier.

Another embodiment of the invention is a method of treating cancer comprising administering to a person in need thereof a therapeutically effective amount of a compound according to formula I.

Another embodiment of the invention is a method of treating colorectal cancer, breast cancer, lung cancer, prostate cancer, pancreatic cancer, gastric cancer, bladder cancer, ovarian cancer, melanoma, neuroblastoma, cervical cancer, kidney cancer or renal cancer, leukemias or lymphomas comprising administering to a person in need thereof a therapeutically effective amount of a compound according to formula I.

### Pharmacological Activity

The compounds of formula I and their pharmaceutically acceptable salts possess valuable pharmacological properties. It has been found that said compounds inhibit the HER-signalling pathway and show anti-proliferative activity. Consequently the compounds of the present invention are useful in the therapy and/or prevention of illnesses with known over-expression of receptor tyrosine kinases of the HER-family like HER-2 and EGFR (HER-1), especially in the therapy and/or prevention of illnesses mentioned above. The activity of the present compounds as antiproliferative inhibitors is demonstrated by the following biological assay:

### CellTiter-Glo™ Assay in HEK293 Cells

The CellTiter-Glo™ Luminescent Cell Viability Assay (Promega) is a homogeneous method of determining the number of viable cells in culture based on quantitation of the ATP present, which signals the presence of metabolically active cells.

HEK293 cells (human embryonic kidney cell line transformed by Adenovirus 5 fragments, ATCC-No. CRL 1573) were cultivated in Dulbecco's Modified Eagle Medium (DMEM) with Glutamax™ (Invitrogen, 31966-021), 5% Fetal Calf Serum (FCS, Sigma Cat-No. F4135 (FBS)), 100 Units/ml penicillin/100 μg/ml streptomycin (=Pen/Strep from Invitrogen Cat. No. 15140). For the assay the cells were seeded in 384 well plates, 5000 cells per well, in the same medium. The next day the test compounds were added in various concentrations ranging from 3 μM to 0.00015 μM (10 concentrations, 1:3 diluted). After 7 days the CellTiter-Glo™ assay was done according to the instructions of the manufacturer (CellTiter-Glo™ Luminescent Cell Viability Assay, from Promega). In brief: the cell-plate was equilibrated to room temperature for approximately 30 minutes and than the CellTiter-Glo™ reagent was added. The contents were carefully mixed for 15 minutes to induce cell lysis. After 45 minutes the luminescent signal was measured in Victor 2, (scanning multiwell spectrophotometer, Wallac).

### Details:

### 1. Day:

Medium: Dulbecco's Modified Eagle Medium (DMEM) with Glutamax™ (Invitrogen, 31966-021), 5% Fetal Calf Serum (FCS, Sigma Cat-No. F4135 (FBS)), Pen/Strep (Invitrogen Cat. No. 15140).
HEK293 (ATCC-No. CRL 1573): 5000 cells in 60 μl per well of 384 well plate (Greiner 781098, white plates)
Incubate 24 h at 37° C., 5% CO₂

### 2. Day: Induction (Substance Testing):

In general the dilution steeps are 1:3
a) Add 8 μl of 10 mM stock solution of compound to 72 μl DMSO
b) dilute 9×1:3 (always 30 μl to 60 μl DMSO) in this DMSO dilution row (results in 10 wells with concentrations from 1000 μM to 0.06 μM)
c) dilute each concentration 1:4.8 (10 μl compound dilution to 38 μl medium)
d) dilute each concentration 1:10 (10 μl compound dilution to 90 μl medium)
e) add 10 μl of every concentration to 60 μl medium in the cell plate
   resulting in final concentration of DMSO: 0.3% in every well
   and resulting in final concentration of compounds from 3 μM to 0.00015 μM
   Incubate 168 h (7 days) at 37° C., 5% CO₂

### Analysis:

Add 30 μl CellTiter-Glo™ Reagent/well,
shake 15 minutes at room temperature
incubate further 45 minutes at room temperature without shaking.

### Measurement:

Victor 2 scanning multiwell spectrophotometer (Wallac), Luminescence mode
Determine IC50 with XL-fit (XLfit software (ID Business Solution Ltd., Guilford, Surrey, UK)).

A significant inhibition of HEK293 cell viability was detected, which is exemplified by the compounds shown in Table 1.

TABLE 1
Results:

| Examples | IC50 HEK293 [nM] |
|---|---|
| 1 | 95 |
| 2, 3, 4, 6, 7, | 5-250 |
| 8, 9 | 250-1000 |

The compounds according to this invention and their pharmaceutically acceptable salts can be used as medicaments, e.g. in the form of pharmaceutical compositions. The pharmaceutical compositions can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions.

The above-mentioned pharmaceutical compositions can be obtained by processing the compounds according to this invention with pharmaceutically acceptable, inorganic or organic carriers. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Pharmaceutical compositions comprise e.g. the following:

### a) Tablet Formulation (Wet Granulation):

| Item | Ingredients | mg/tablet | | | |
|---|---|---|---|---|---|
| 1. | Compound of formula (I) | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure:

1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

### b) Capsule Formulation:

| Item | Ingredients | mg/capsule | | | |
|---|---|---|---|---|---|
| 1. | Compound of formula (I) | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

### Manufacturing Procedure:

1. Mix items 1, 2 and 3 in a suitable mixer for 30 minutes.
2. Add items 4 and 5 and mix for 3 minutes.
3. Fill into a suitable capsule.

The following examples and references are provided to aid the understanding of the present invention, the true scope of which is set forth in the appended claims. It is understood that modifications can be made in the procedures set forth without departing from the spirit of the invention.

### Experimental Procedures

Intermediate 1

### 2-(2-Methyl-2H-pyrazol-3-yl)-ethanol

### a) 5-[2-(tert-Butyl-dimethyl-silanyloxy)-ethyl]-1-methyl-1H-pyrazole

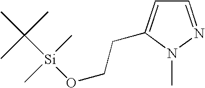

To 10.00 g (0.122 mol) 1-methyl-1H-pyrazole in 200 ml dry tetrahydrofuran (THF) under a nitrogen atmosphere at −65° C. was added 53.6 ml (0.134 mol) n-Butyllithium in hexane (2.5 M). The mixture was stirred 1 h at −65° C. Then 29.14 g (0.122 mol) (2-bromethoxy)-tert-butyl-dimethylsilane were added slowly. After stirring of the reaction for 2 h, it was allowed to warm up to room temperature and stirring was continued overnight. Water was added and the THF was removed in vacuo. The aqueous layer was neutralized with 1N HCl and extracted several times with ethyl acetate. The organic phases were collected, dried over Na₂SO₄ and the solvent was removed in vacuo. Flash chromatography (silica, 30% ethyl acetate in n-heptane) yielded 6.30 g (22%) 5-[2-(tert-Butyl-dimethyl-silanyloxy)-ethyl]-1-methyl-1H-pyrazole as a colorless oil.

¹H-NMR (400 MHz, D₆-DMSO): δ=0.00 (s, 6H), 0.85 (s, 9H), 2.82 (t, 6.5 Hz, 2H), 3.75 (s, 3H), 3.80 (t, 6.5 Hz, 2H), 6.06 (d, 1.5 Hz, 1H), 7.28 (d, 1.5 Hz, 1H)

### b) 2-(2-Methyl-2H-pyrazol-3-yl)-ethanol

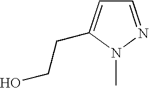

To a solution of 12.00 g (0.05 mol) 5-[2-(tert-Butyl-dimethyl-silanyloxy)-ethyl]-1-methyl-pyrazole in 150 ml N,N-dimethylformamide (DMF) were added 6.29 g (0.150 mol) sodium fluoride and 17.0 ml (0.150 mol) aqueous 48% HBr and the mixture was stirred overnight at room temperature. All volatiles were removed in vacuo and the residue was taken up in ethyl acetate/water. The aqueous layer was neutralized with Na₂CO₃, saturated with NaCl and extracted three times with ethyl acetate. The combinrd organic layers were collected, washed with saturated aqueous NaCl, dried over Na₂SO₄ and the solvent was removed in vacuo. Flash chromatography (silica, ethyl acetate) yielded 3.80 g (61%) 2-(2-Methyl-2H-pyrazol-3-yl)-ethanol as a yellow oil.

¹H-NMR (400 MHz, D₆-DMSO): δ=2.75 (t, 6.9 Hz, 2H), 3.61 (t, 6.9 Hz, 2H), 3.72 (s, 3H), 6.04 (d, 1.5 Hz, 1H), 7.26 (s, 1.5 Hz, 1H)

### Intermediate 2

### 3-Methyl-4-[2-(2-methyl-2H-pyrazol-3-yl)-ethoxymethyl]-phenol

### a) 4-Allyloxy-2-methyl-benzaldehyde

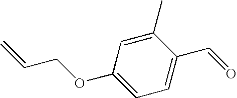

31.7 g (229 mmol) potassium carbonate and 9.51 g (57.3 mmol) potassium iodide were given to a solution of 15.6 g (115 mmol) 4-hydroxy-2-methyl-benzaldehyde and 55.4 g (458 mmol) allyl bromide in 500 ml 2-butanone and stirred for 16 h at 65° C. Solvents were distilled off and the residue distributed between ethyl acetate and 1 N sodium hydroxide. The organic layer was separated and the aqueous solution extracted once with ethyl acetate. The combined organic phases were dried and evaporated to give 19.8 g (98%) of 4-allyloxy-2-methyl-benzaldehyde.

¹H-NMR (400 MHz, D₆-DMSO): δ=2.59 (s, 3H), 4.67 (d, 2H), 5.29 (d, 1H), 5.41 (d, 1H), 6.05 (m, 1H), 6.96 (d, 1H), 6.74 (s, 1H), 7.77 (d, 1H), 10.07 (s, 1H). CL b) (4-Allyloxy-2-methyl-phenyl)-methanol

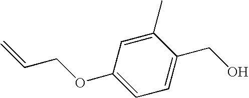

8.50 g (224 mmol) lithium aluminium hydride were given to 250 ml tetrahydrofuran (THF) and stirred for 20 min. A solution of 19.4 g (110 mmol) 4-allyloxy-2-methyl-benzaldehyde in 100 ml THF was added dropwise and stirring continued for 3 h. The reaction mixture was cooled to 0° C., carefully hydrolysed with 40 ml concentrated ammonium chloride solution, stirred for 60 min. and adjusted to pH=5 with conc. hydrochloric acid. A formed salt precipitate was removed by filtration, washed with THF and the combined organic solutions evaporated. Chromatography of the residue on silica (n-heptane/ethyl acetate 1:3) gave 16.0 g (81%) (4-allyloxy-2-methyl-phenyl)-methanol as a slightly yellow oil.

¹H-NMR (400 MHz, D₆-DMSO): δ=2.23 (s, 3H), 4.40 (s, 2H), 4.52 (d, 2H), 4.88 (t, 1H), 5.23 (d, 1H), 5.37 (d, 1H), 6.03 (m, 1H), 6.72 (d, 1H), 6.74 (s, 1H), 7.20 (d, 1H).

### c) 4-Allyloxy-1-chloromethyl-2-methyl-benzene

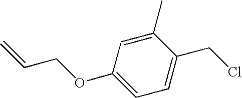

A solution of 16.0 g (89.6 mmol) (4-allyloxy-2-methyl-phenyl)-methanol in 270 ml dichloromethane and 1.5 ml N,N-dimethylformamide (DMF) was cooled to 0° C. 7.80 ml (12.8 g, 108 mmol) thionyl chloride were added slowly and then stirred for 1 h at room temperature. Dichloromethane was distilled off, 300 ml toluene added and solvents removed in vacuo. The residue was taken up in 200 ml toluene and washed with concentrated sodium carbonate solution. The organic phase was dried and evaporated to give 17.5 g (99%) 1-allyloxy-4-chloromethyl-2-methyl-benzene as colored oil.

¹H-NMR (400 MHz, D₆-DMSO): δ=2.34 (s, 3H), 4.74 (d, 2H), 4.55 (s, 2H), 5.25 (d, 1H), 5.38 (d, 1H), 6.02 (m, 1H), 6.75 (d, 1H), 6.82 (s, 1H), 7.29 (d, 1H).

### d) 5-[2-(4-Allyloxy-2-methyl-benzyloxy)-ethyl]-1-methyl-1H-pyrazole

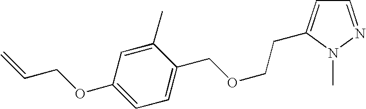

0.856 g (0.036 mmol) 95% sodium hydride were given at −50° C. to a solution of 4.677 g (0.024 mmol) 1-allyloxy-4-chloromethyl-2-methyl-benzene and 3.000 g (0.024 mmol) 2-(2-Methyl-2H-pyrazol-3-yl)-ethanol in N,N-dimethylformamide (DMF). The mixture was allowed to warm slowly to r. t., stirred for 5 hours. The mixture was concentrated in vacuo and the residue was partitionated between ethyl acetate and water. The organic layer was dried over Na₂SO₄ and the solvent was distilled off under reduced pressure to yield 6.40 g (94%)-[2-(4-Allyloxy-2-methyl-benzyloxy)-ethyl]-1-methyl-1H-pyrazole as brown oil which was used without further purification.

¹H-NMR (400 MHz, D₆-DMSO): δ=2.20 (s, 3H), 2.88 (t, 6.6 Hz, 2H), 3.63 (t, 6.6 Hz, 2H), 3.70 (s, 3H), 4.40 (s, 2H), 4.51 (m, 2H), 5.23 (m, 1H), 5.37 (m, 1H), 6.02 (d, 1.8 Hz, 1H), 6.06 (m, 1H), 6.71 (dd, 2.6 Hz, 8.3 Hz, 1H), 6.77 (d, 2.6 Hz, 1H,), 7.15 (d, 8.3 Hz, 1H,), 7.26 (d, 1.8 Hz, 1H)

### e) 3-Methyl-4-[2-(2-methyl-2H-pyrazol-3-yl)-ethoxymethyl]-phenol

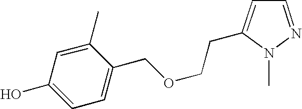

A solution of 6.40 g (22.4. mmol)-[2-(4-Allyloxy-2-methyl-benzyloxy)-ethyl]-1-methyl-1H-pyrazole in 100 ml dichloromethane was added to a solution of 10.47 g (67.0 mmol) 1,3-dimethylbarbituric acid an 774 mg (0.7 mmol) Pd(PPh₃)₄ in 30 ml dichloromethane and stirred overnight at 40° C. The mixture was extracted with 3×sat. NaHCO₃-solution. The combined aqueous phases were extracted with dichloromethane. The combined organic extracts were dried over Na₂SO₄. Solvents were distilled off and the residue was purified by chromatography on silica gel (heptane/ethyl acetate 1/1) to yield 1.60 g (29%) 3-Methyl-4-[2-(2-methyl-2H-pyrazol-3-yl)-ethoxymethyl]-phenol as a orange solid.

¹H-NMR (400 MHz, D₆-DMSO): δ=2.15 (s, 3H), 2.86 (t, 6.6 Hz, 2H), 3.61 (t, 6.6 Hz, 2H), 4.35 (s, 2H), 6.02 (d, 1.8 Hz, 1H), 6.51 (dd, 2.4 Hz, 8.1 Hz, 1H), 6.57 (d, 2.4 Hz, 1H,), 7.02 (d, 8.1 Hz, 1H,), 7.26 (d, 1.8 Hz, 1H), 9.24 (s, 1H)

### Example 1

### 4-{3-Methyl-4-[2-(E)-(2-methyl-2H-pyrazol-3-yl)-ethoxymethyl]-phenoxymethyl}-2-[2-(4-trifluoromethoxy-phenyl)-vinyl]-oxazole

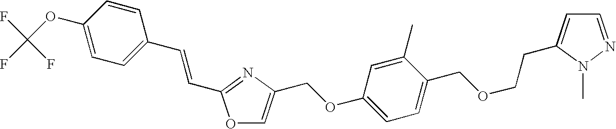

51 mg (2.11 mmol) 95% sodium hydride were given to a solution of 259 mg (1.05 mmol) 3-Methyl-4-[2-(2-methyl-2H-pyrazol-3-yl)-ethoxymethyl]-phenol in 20 ml N,N-dimethylformamide (DMF) and stirred for 15 minutes. Then 320 mg (1.05 mmol) 4-chloromethyl-2-[2-(4-trifluoromethoxy-phenyl)-vinyl]-oxazole were added and stirring continued at room temperature overnight. After addition of 5 ml water all valatiles were removed in vacuo and the residue was purified by HPLC/MS (RP 18, methanol-water-gradient) to yield 300 mg (56%) 4-{3-Methyl-4-[2-(E)-(2-methyl-2H-pyrazol-3-yl)-ethoxymethyl]-phenoxymethyl}-2-[2-(4-trifluoromethoxy-phenyl)-vinyl]-oxazole as a colorless solid melting at 77-78° C.

MS: M=514.3 (ES+)

¹H-NMR (400 MHz, D₆-DMSO): δ=2.22 (s, 3H), 2.89 (t, 6.6 Hz, 2H), 3.64 (t, 6.6 Hz, 2H), 3.71 (s, 3H), 4.42 (s, 2H), 5.00 (s, 2H), 6.03 (s, 1H), 6.82 (dd, 2.5 Hz, 8.0 Hz, 1H), 6.86 (d, 2.5 Hz, 1H), 7.18 (d, 8.0 Hz, 1H), 7.25 (d, 16.4 Hz, 1H), 7.26 (s, 1H), 7.40 (d, 8.5 Hz, 2H), 7.56 (d, 16.4 Hz, 1H), 7.87 (d, 8.5 Hz, 2H), 8.21 (s, 1H)

### Example 2

### 4-{3-Methyl-4-[2-(E)-(2-methyl-2H-pyrazol-3-yl)-ethoxymethyl]-phenoxymethyl}-2-[2-(4-trifluoromethyl-phenyl)-vinyl]-oxazole

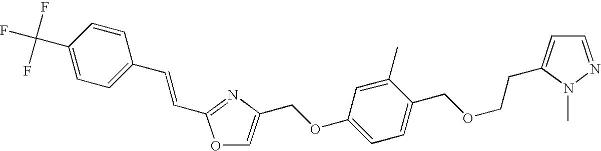

An analogous reaction to that described in example 1 using 4-chloromethyl-2-[2-(4-trifluoromethyl-phenyl)-vinyl]-oxazole yielded 77% 4-{3-Methyl-4-[2-(E)-(2-methyl-2H-pyrazol-3-yl)-ethoxymethyl]-phenoxymethyl}-2-[2-(4-trifluoromethyl-phenyl)-vinyl]-oxazole as white solid melting at 96-97° C.

MS: M=498.4 (ES+)

¹H-NMR (400 MHz, D₆-DMSO): δ=2.21 (s, 3H), 2.89 (t, 6.6 Hz, 2H), 3.64 (t, 6.6 Hz, 2H), 3.71 (s, 3H), 4.42 (s, 2H), 4.99 (s, 2H), 6.03 (s, 1H), 6.82 (dd, 2.5 Hz, 8.3 Hz, 1H), 6.86 (d, 2.5 Hz, 1H), 7.18 (d, 8.3 Hz, 1H), 7.26 (s, 1H), 7.34 (d, 16.4 Hz, 1H), 7.62 (d, 16.4 Hz, 1H), 7.76 (d, 8.3 Hz, 2H), 7.95 (d, 8.3 Hz, 2H), 8.24 (s, 1H)

### Example 3

### 2-[2-(E)-(4-Chloro-phenyl)-vinyl]-4-{3-methyl-4-[2-(2-methyl-2H-pyrazol-3-yl)-ethoxymethyl]-phenoxymethyl}-oxazole

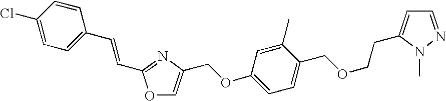

An analogous reaction to that described in example 1 using 4-chloromethyl-2-[2-(4-chloro-phenyl)-vinyl]-oxazole yielded 50% 4-{3-Methyl-4-[2-(E)-(2-methyl-2H-pyrazol-3-yl)-ethoxymethyl]-phenoxymethyl}-2-[2-(4-trifluoromethyl-phenyl)-vinyl]-oxazole as white solid melting at 106-107° C.

MS: M=464.6 (ES+)

¹H-NMR (400 MHz, D₆-DMSO): δ=2.21 (s, 3H), 2.88 (t, 6.6 Hz, 2H), 3.64 (t, 6.6 Hz, 2H), 3.71 (s, 3H), 4.42 (s, 2H), 4.99 (s, 2H), 6.04 (d, 1.6 Hz, 1H), 6.81 (dd, 2.4 Hz, 8.2 Hz, 1H), 6.86 (d, 2.4 Hz, 1H), 7.19 (d, 16.4 Hz, 1H), 7.20 (d, 8.2 Hz, 1H), 7.26 (d, 1.6 Hz, 1H), 7.47 (d, 8.3 Hz, 2H), 7.52 (d, 16.4 Hz, 1H), 7.76 (d, 8.3 Hz, 2H), 8.20 (s, 1H)

### Example 4

### 2-[2-(E)-(2-Fluoro-4-trifluoromethyl-phenyl)-vinyl]-4-{3-methyl-4-[2-(2-methyl-2H-pyrazol-3-yl)-ethoxymethyl]-phenoxymethyl}-oxazole

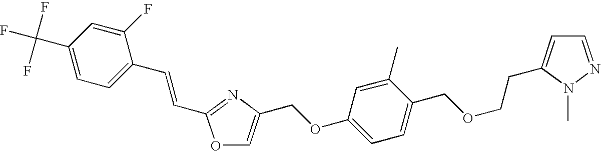

An analogous reaction to that described in example 1 using 4-chloromethyl-2-[2-(2-fluoro-4-trifluoromethyl-phenyl)-vinyl]-oxazole yielded 61% 4-{3-Methyl-4-[2-(E)-(2-methyl-2H-pyrazol-3-yl)-ethoxymethyl]-phenoxymethyl}-2-[2-(4-trifluoromethyl-phenyl)-vinyl]-oxazole as pale yellow solid.

MS: M=516.4 (ES+)

¹H-NMR (400 MHz, D₆-DMSO): δ=2.22 (s, 3H), 2.89 (t, 6.6 Hz, 2H), 3.65 (t, 6.6 Hz, 2H), 3.71 (s, 3H), 4.42 (s, 2H), 5.01 (s, 2H), 6.04 (d, 1.8 Hz, 1H), 6.82 (dd, 2.7 Hz, 8.3 Hz, 1H), 6.86 (d, 2.7 Hz, 1H), 7.18 (d, 8.3 Hz, 1H), 7.26 (d, 1.8 Hz, 1H), 7.39 (d, 16.4 Hz, 1H), 7.59 (d, 16.4 Hz, 1H), 7.64 (d, 7.6 Hz, 1H), 7.77 (d, 10.9 Hz, 1H), 8.15 (t, 7.9 Hz, 1H), 8.27 (s, 1H)

### Intermediate 3

### Toluene-4-sulfonic acid 2-(2-methyl-2H-pyrazol-3-yl)-ethyl ester

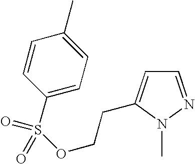

To a solution of 5.74 g (30.1 mmol) toluene-4-sulfonylchloride, 3.69 g (36.5 mmol) triethylamine and 0.92 g (7.5 mmol) 4-(N,N-dimethylamino)-pyridine (DMAP) in 75 ml dichloromethane was dropped at −10° C. a solution of 3.8 g (30.1 mmol) 2-(2-Methyl-2H-pyrazol-3-yl)-ethanol in 75 ml dichloromethane and stirring continued at −4° C. overnight. After addition of 100 ml ice water and 100 ml dichloromethane the phases were separated and the organic layer was washed with sodium bicarbonate solution, dried and evaporated. Yield: 6.81 g (81%) Toluene-4-sulfonic acid 2-(2-methyl-2H-pyrazol-3-yl)-ethyl ester as a yellow liquid which was used without further purification.

¹H-NMR (400 MHz, D₆-DMSO): δ=2.42 (s, 3H), 2.99 (t, 6.2 Hz, 2H), 3.66 (s, 3H), 4.24 (t, 6.2 Hz, 2H), 5.99 (d, 1.5 Hz, 1H), 7.27 (d, 1.5 Hz, 1H), 7.47 (d, 8.3 Hz, 2H), 7.74 (d, 8.3 Hz, 2H)

### Intermediate 4

### 3-Methyl-4-[2-(2-methyl-2H-pyrazol-3-yl)-ethanesulfinylmethyl]-phenol

### a) (4-Allyloxy-2-methyl-phenyl)-methanethiol

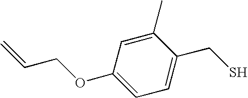

A mixture of 19.6 g (107.3 mmol) 1-allyloxy-4-chloromethyl-benzene and 8.99 g (118 mmol) thiourea in 25 ml ethanol was refluxed for 7 hours, then allowed to cool over night, evaporated and the residue washed with ethanol. This was heated to reflux with 25 ml ethanol and 7.5 ml 25% ammonia for 2 hours, then evaporated and partitioned between 5 ml 6N HCl and ethyl acetate. The organic phase was dried and evaporated to leave 13.65 g (71%) (4-allyloxy-phenyl)-methanethiol as nearly colourless oil.

¹H-NMR (400 MHz, D₆-DMSO): δ=2.30 (s, 3H), 2.60 (t, 7.1 Hz, 1H), 3.67 (d, 7.1 Hz, 2H), 4.52 (m, 2H), 5.24 (m, 1H), 5.37 (m, 1H), 6.02 (m, 1H), 6.71 (dd, 2.7 Hz, 8.3 Hz, 1H), 6.76 (d, 2.7 Hz, 1H), 7.15 (d, 8.3 Hz, 1H)

### b) 5-[2-(4-Allyloxy-2-methyl-benzylsulfanyl)-ethyl]-1-methyl-1H-pyrazole

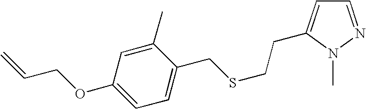

To a mixture of 4.75 g (24.5 mmol) (4-Allyloxy-2-methyl-phenyl)-methanethiol and 6.86 g (24.5 mmol) toluene-4-sulfonic acid 2-(2-methyl-2H-pyrazol-3-yl)-ethyl ester in 30 ml N,N-dimethylformamide was added under argon at −30° C. 0.71 g (29.4 mmol) sodium hydride. The mixture was allowed to warm up to room temperature and was stirred under argon for 12 hours. After quenching with 100 ml water, the mixture was diluted with dichloromethane, washed with water, dried and evaporated. Purification on silica after elution with ethyl acetate/heptane 1:1 yielded 5.30 g (72%) 5-[2-(4-Allyloxy-2-methyl-benzylsulfanyl)-ethyl]-1-methyl-1H-pyrazole as slightly yellow oil.

¹H-NMR (400 MHz, D₆-DMSO): δ=2.31 (s, 3H), 2.67 (t, 7.6 Hz, 2H), 2.85 (t, 7.6 Hz, 2H), 3.71 (s, 3H), 3.72 (s, 2H), 4.52 (m, 2H), 5.24 (m, 1H), 5.37 (m, 1H), 6.02 (m, 1H), 6.04 (d, 1.7 Hz, 1H), 6.71 (dd, 2.7 Hz, 8.3 Hz, 1H), 6.78 (d, 2.7 Hz, 1H), 7.12 (d, 8.3 Hz, 1H), 7.27 (d, 1.7 Hz, 1H)

### c) 5-[2-(4-Allyloxy-2-methyl-phenylmethanesulfinyl)-ethyl]-1-methyl-1H-pyrazole

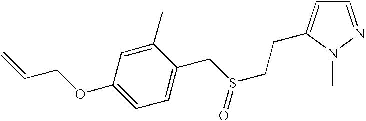

To a solution of 5.35 g (17.7 mmol) 5-[2-(4-Allyloxy-2-methyl-benzylsulfanyl)-ethyl]-1-methyl-1H-pyrazole in 150 ml dichloromethane was added dropwise at −30° C. a solution of 3.05 g (17.7 mmol) 3-chloro-benzenecarboperoxoic acid in dichloromethane and stirring continued for 1 hour. The mixture was allowed to warm up over night, washed with sodium bicarbonate and sodium carbonate solution, then with water, dried and evaporated. Elution form silica with ethyl acetate/heptane (1/1 to 1/0) furnished 4.44 g (71%) 5-[2-(4-Alyloxy-2-methyl-phenylmethanesulfinyl)-ethyl]-1-methyl-1H-pyrazole as white solid.

¹H-NMR (400 MHz, D₆-DMSO): δ=2.31 (s, 3H), 2.93-3.16 (m, 4H), 3.75 (s, 3H), 4.00 (d, 13.1 Hz, 1H), 4.15 (d, 13.1 Hz, 1H), 4.55 (m, 2H), 5.24 (m, 1H), 5.37 (m, 1H), 6.02 (m, 1H), 6.09 (d, 1.5 Hz, 1H), 6.77 (dd, 2.5 Hz, 8.3 Hz, 1H), 6.83 (d, 2.5 Hz, 1H), 7.17 (d, 8.3 Hz, 1H), 7.30 (d, 1.7 Hz, 1H)

### d) 3-Methyl-4-[2-(2-methyl-2H-pyrazol-3-yl)-ethanesulfinylmethyl]-phenol

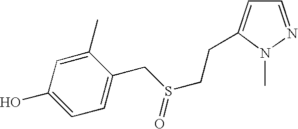

To a solution of 5.88 g (0.038 mol) 1,3-dimethyl-pyrimidine-2,4,6-trione and 0.44 g (0.38 mmol) tetrakis-(triphenylphosphine)-palladium in 100 ml dichloromethane was added dropwise a solution of 4.00 g (12.56 mmol) 5-[2-(4-Allyloxy-2-methyl-phenylmethanesulfinyl)-ethyl]-1-methyl-1H-pyrazole and stirring was continued overnight at 45° C. The reaction mixture was extracted with three portions of sodium bicarbonate solution The organic layer was dried and evaporated. HPLC/MS (RP 18, methanol-water-gradient) yielded 2.20 g (63%) 3-Methyl-4-[2-(2-methyl-2H-pyrazol-3-yl)-ethanesulfinylmethyl]-phenol as yellow solid.

¹H-NMR (400 MHz, D₆-DMSO): δ=2.26 (s, 3H), 2.91-3.14 (m, 4H), 3.75 (s, 3H), 3.96 (d, 13.1 Hz, 1H), 4.10 (d, 13.1 Hz, 1H), 6.09 (d, 1.8 Hz, 1H), 6.58 (dd, 2.4 Hz, 8.3 Hz, 1H), 6.63 (d, 2.4 Hz, 1H), 7.06 (d, 8.3 Hz, 1H), 7.31 (d, 1.8 Hz, 1H), 9.39 (s, 1H)

### Example 5

### 4-{3-Methyl-4-[2-(2-methyl-2H-pyrazol-3-yl)-ethanesulfinylmethyl]-phenoxymethyl}-2-[2-(E)-(4-trifluoromethoxy-phenyl)-vinyl]-oxazole

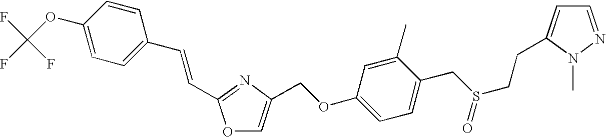

22 mg (0.91 mmol) 95% sodium hydride were given to a solution of 212 mg (0.76 mmol) 4-[2-(2-Methyl-2H-pyrazol-3-yl)-ethanesulfinylmethyl]-phenol in 20 ml N,N-dimethylformamide (DMF) and stirred for 30 minutes. Then 230 mg (0.76 mmol) 4-chloromethyl-2-[2-(4-trifluoromethoxy-phenyl)-vinyl]-oxazole were added and stirring continued at room temperature overnight. After addition of 5 ml water all volatiles were removed in vacuo and the residue was purified by HPLC/MS (RP 18, methanol-water-gradient) to yield 170 mg (41%) 4-{4-[2-(2-Methyl-2H-pyrazol-3-yl)-ethanesulfinylmethyl]-phenoxymethyl}-2-[2-(E)-(4-trifluoromethoxy-phenyl)-vinyl]-oxazole as a colorless solid melting at 138-139° C.

MS: M=546.4 (ES+)

¹H-NMR (400 MHz, D₆-DMSO): δ=2.33 (s, 3H), 2.93-3.16 (m, 4H), 3.76 (s, 3H), 4.02 (d, 13.1 Hz, 1H), 4.17 (d, 13.1 Hz, 1H), 5.01 (s, 2H), 6.10 (d, 1.8 Hz, 1H), 6.88 (dd, 2.5 Hz, 8.1 Hz, 1H), 6.92 (d, 2.5 Hz, 1H), 7.20 (d, 8.1 Hz, 1H), 7.21 (d, 16.4 Hz, 1H), 7.30 (d, 1.8 Hz, 1H), 7.40 (d, 8.5 Hz, 2H), 7.57 (d, 16.4 Hz, 1H), 7.87 (d, 8.5 Hz, 2H), 8.22 (s, 1H)

### Example 6

### 2-[2-(E)-(4-Chloro-phenyl)-vinyl]-4-{3-methyl-4-[2-(2-methyl-2H-pyrazol-3-yl)-ethanesulfinylmethyl]-phenoxymethyl}-oxazole

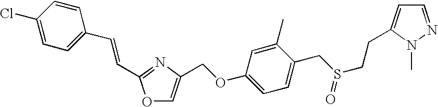

An analogous reaction to that described in example 8 using 4-chloromethyl-2-[2-(4-chloromethyl-phenyl)-vinyl]-oxazole yielded 31% 2-[2-(E)-(4-Chloro-phenyl)-vinyl]-4-{3-methyl-4-[2-(2-methyl-2H-pyrazol-3-yl)-ethanesulfinylmethyl]-phenoxymethyl}-oxazole as a white solid melting at 169-171° C.

MS: M=496.4 (ES+)

¹H-NMR (400 MHz, D₆-DMSO): δ=2.33 (s, 3H), 2.93-3.17 (m, 4H), 3.75 (s, 3H), 4.01 (d, 12.9 Hz, 1H), 4.16 (d, 12.9 Hz, 1H), 5.01 (s, 2H), 6.09 (d, 1.8 Hz, 1H), 6.88 (dd, 2.5 Hz, 8.1 Hz, 1H), 6.92 (d, 2.5 Hz, 1H), 7.20 (d, 16.4 Hz, 1H), 7.20 (d, 8.1 Hz, 1H), 7.30 (d, 1.8 Hz, 1H), 7.47 (d, 8.5 Hz, 2H), 7.53 (d, 16.4 Hz, 1H), 7.76 (d, 8.5 Hz, 2H), 8.21 (s, 1H)

### Example 7

### 4-{3-Methyl-4-[2-(2-methyl-2H-pyrazol-3-yl)-ethanesulfinylmethyl]-phenoxymethyl}-2-[2-(E)-(4-trifluoromethyl-phenyl)-vinyl]-oxazole

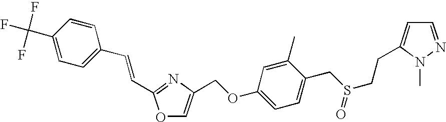

An analogous reaction to that described in example 8 using 4-chloromethyl-2-[2-(4-trifluoromethyl-phenyl)-vinyl]-oxazole yielded 30% 4-{3-Methyl-4-[2-(2-methyl-2H-pyrazol-3-yl)-ethanesulfinylmethyl]-phenoxymethyl}-2-[2-(E)-(4-trifluoromethyl-phenyl)-vinyl]-oxazole as a white solid melting at 161-162° C.

MS: M=530.3 (ES+)

¹H-NMR (400 MHz, D₆-DMSO): δ=2.33 (s, 3H), 2.91-3.16 (m, 4H), 3.76 (s, 3H), 4.02 (d, 13.0 Hz, 1H), 4.17 (d, 13.0 Hz, 1H), 5.03 (s, 2H), 6.10 (s, 1H), 6.89 (dd, 2.5 Hz, 8.1 Hz, 1H), 6.92 (d, 2.5 Hz, 1H), 7.20 (d, 8.1 Hz, 1H), 7.30 (s, 1H), 7.34 (d, 16.4 Hz, 1H), 7.62 (d, 16.4 Hz, 1H), 7.77 (d, 8.3 Hz, 2H), 7.96 (d, 8.3 Hz, 2H), 8.26 (s, 1H)

### Intermediate 5

### 4-[2-(2-Methyl-2H-pyrazol-3-yl)-ethanesulfinylmethyl]-phenol

### a) (4-Allyloxy-phenyl)-methanethiol

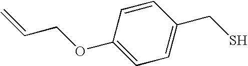

A mixture of 19.6 g (107.3 mmol) 1-allyloxy-4-chloromethyl-benzene and 8.99 g (118 mmol) thiourea in 25 ml ethanol was refluxed for 7 hours, then allowed to cool over night, evaporated and the residue washed with ethanol. This was heated to reflux with 25 ml ethanol and 7.5 ml 25% ammonia for 2 hours, then evaporated and partitioned between 5 ml 6N HCl and ethyl acetate. The organic phase was dried and evaporated to leave 13.65 g (71%) (4-allyloxy-phenyl)-methanethiol as nearly colourless oil.

¹H-NMR (400 MHz, D₆-DMSO): δ=2.73 (t, 7.4 Hz, 1H), 3.68 (d, 7.4 Hz, 2H), 4.53 (m, 2H), 5.24 (m, 1H), 5.38 (m, 1H), 6.03 (m, 1H), 6.88 (d, 8.6 Hz, 2H), 7.24 (d, 8.6 Hz, 2H)

### b) 5-[2-(4-Allyloxy-benzylsulfanyl)-ethyl]-1-methyl-1H-pyrazole

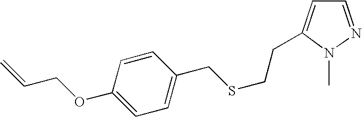

To a mixture of 2.87 g (15.9 mmol) (4-allyloxy-phenyl)-methanethiol and 4.46 g (15.9 mmol) toluene-4-sulfonic acid 2-(2-methyl-2H-pyrazol-3-yl)-ethyl ester in 30 ml N,N-dimethylformamide (DMF) was added under argon at −30° C. 0.46 g (19.1 mmol) sodium hydride. The mixture was allowed to warm up to room temperature and was stirred under argon for 12 hours. After quenching with 100 ml water, the mixture was diluted with dichloromethane, washed with water, dried and evaporated. Purification on silica after elution with ethyl acetate/heptane 1:1 yielded 2.25 g (49%)-[2-(4-Allyloxy-benzylsulfanyl)-ethyl]-1-methyl-1H-pyrazole as slightly yellow oil.

¹H-NMR (400 MHz, D₆-DMSO): δ=2.62 (t, 7.6 Hz, 2H), 2.83 (t, 7.6 Hz, 2H), 3.69 (s, 3H), 3.72 (s, 2H), 4.54 (m, 2H), 5.24 (m, 1H), 5.38 (m, 1H), 6.02 (m, 1H), 6.03 (d, 1.8 Hz, 1H), 6.89 (d, 8.6 Hz, 2H), 7.23 (d, 8.6 Hz, 2H), 7.26 (d, 1.8 Hz, 1H)

### c) 5-[2-(4-Allyloxy-phenylmethanesulfinyl)-ethyl]-1-methyl-1H-pyrazole

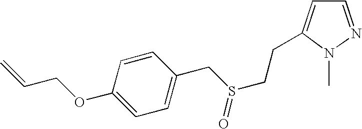

To a solution of 2.26 g (7.8 mmol) 5-[2-(4-Allyloxy-benzylsulfanyl)-ethyl]-1-methyl-1H-pyrazole in 150 ml dichloromethane was added dropwise at −30° C. a solution of 1.76 g (77%, 7.8 mmol) 3-chloro-benzenecarboperoxoic acid in dichloromethane and stirring continued for 1 hour. The mixture was allowed to warm up over night, washed with sodium bicarbonate and sodium carbonate solution, then with water, dried and evaporated. Elution form silica with ethyl acetate/heptane (1/1 to 1/0) furnished 5-[2-(4-Allyloxy-phenylmethanesulfinyl)-ethyl]-1-methyl-1H-pyrazole as white solid in quantitative yield.

¹H-NMR (400 MHz, D₆-DMSO): δ=2.78-3.08 (m, 4H), 3.74 (s, 3H), 3.95 (d, 12.9 Hz, 1H), 4.12 (d, 12.9 Hz, 1H), 4.56 (m, 2H), 5.26 (m, 1H), 5.39 (m, 1H), 6.04 (m, 1H), 6.08 (d, 1.8 Hz, 1H), 6.95 (d, 8.6 Hz, 2H), 7.24 (d, 8.6 Hz, 2H), 7.29 (d, 1.7 Hz, 1H)

### d) 4-[2-(2-Methyl-2H-pyrazol-3-yl)-ethanesulfinylmethyl]-phenol

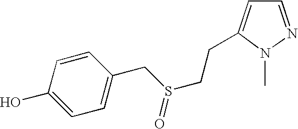

To a solution of 3.85 g (24.6 mmol) 1,3-dimethyl-pyrimidine-2,4,6-trione and 0.289 g (0.25 mmol) tetrakis-(triphenylphosphine)-palladium in 80 ml dichloromethane was added dropwise a solution of 2.50 g (8.21 mmol) 5-[2-(4-allyloxy-phenylmethanesulfinyl)-ethyl]-1-methyl-1H-pyrazole and stirring was continued at 45° C. overnight. The reaction mixture was extracted with three portions of sodium bicarbonate solution The organic layer was dried and evaporated. HPLC/MS (RP 18, methanol-water-gradient) yielded 0.500 g (23%) 4-[2-(2-Methyl-2H-pyrazol-3-yl)-ethanesulfinylmethyl]-phenol as white solid.

¹H-NMR (400 MHz, D₆-DMSO): δ=2.78-3.05 (m, 4H), 3.74 (s, 3H), 3.90 (d, 12.9 Hz, 1H), 4.06 (d, 12.9 Hz, 1H), 6.07 (d, 1.7 Hz, 1H), 6.75 (d, 8.3 Hz, 2H), 7.13 (d, 8.3 Hz, 2H), 7.29 (d, 1.7 Hz, 1H), 9.47 (s, 1H)

### Example 8

### 4-{4-[2-(2-Methyl-2H-pyrazol-3-yl)-ethanesulfinylmethyl]-phenoxymethyl}-2-[2-(E)-(4-trifluoromethoxy-phenyl)-vinyl]-oxazole

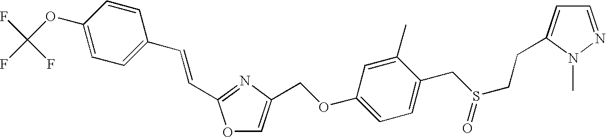

23 mg (0.95 mmol) 95% sodium hydride were given to a solution of 209 mg (0.79 mmol) 4-[2-(2-Methyl-2H-pyrazol-3-yl)-ethanesulfinylmethyl]-phenol in 30 ml N,N-dimethylformamide (DMF) and stirred for 30 minutes. Then 240 mg (0.79 mmol) 4-chloromethyl-2-[2-(4-trifluoromethoxy-phenyl)-vinyl]-oxazole were added and stirring continued at room temperature overnight. After addition of 5 ml water all volatiles were removed in vacuo and the residue was purified by HPLC/MS (RP 18, methanol-water-gradient) to yield 140 mg (33%) 4-{4-[2-(2-Methyl-2H-pyrazol-3-yl)-ethanesulfinylmethyl]-phenoxymethyl}-2-[2-(E)-(4-trifluoromethoxy-phenyl)-vinyl]-oxazole as a colorless solid melting at 144-146° C.

MS: M=532.2 (ES+)

¹H-NMR (400 MHz, D₆-DMSO): δ=2.80-2.92 (m, 2H), 2.96-3.08 (m, 2H), 3.74 (s, 3H), 3.96 (d, 12.9 Hz, 1H), 4.14 (d, 12.9 Hz, 1H), 5.03 (s, 2H), 6.08 (d, 1.8 Hz, 1H), 7.05 (d, 8.6 Hz, 2H), 7.21 (d, 16.4 Hz, 1H), 7.27 (d, 8.6 Hz, 2H), 7.29 (d, 1.8 Hz, 1H), 7.40 (d, 8.5 Hz, 2H), 7.57 (d, 16.4 Hz, 1H), 7.87 (d, 8.5 Hz, 2H), 8.23 (s, 1H)

### Example 9

### 4-{4-[2-(2-Methyl-2H-pyrazol-3-yl)-ethanesulfinylmethyl]-phenoxymethyl}-2-[2-(E)-(4-trifluoromethyl-phenyl)-vinyl]-oxazole

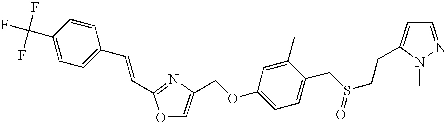

An analogous reaction to that described in example 8 using 4-chloromethyl-2-[2-(4-trifluoromethyl-phenyl)-vinyl]-oxazole yielded 37% 4-{4-[2-(2-Methyl-2H-pyrazol-3-yl)-ethanesulfinylmethyl]-phenoxymethyl}-2-[2-(E)-(4-trifluoromethyl-phenyl)-vinyl]-oxazole as a white solid melting at 172-174° C.

MS: M=516.3 (ES+)

¹H-NMR (400 MHz, D₆-DMSO): δ=2.80-2.92 (m, 2H), 2.96-3.08 (m, 2H), 3.74 (s, 3H), 3.97 (d, 12.9 Hz, 1H), 4.14 (d, 12.9 Hz, 1H), 5.04 (s, 2H), 6.08 (d, 2.0 Hz, 1H), 7.06 (d, 8.6 Hz, 2H), 7.28 (d, 8.6 Hz, 2H), 7.29 (d, 2.0 Hz, 1H), 7.34 (d, 16.4 Hz, 1H), 7.62 (d, 16.4 Hz, 1H), 7.76 (d, 8.5 Hz, 2H), 7.95 (d, 8.5 Hz, 2H), 8.26 (s, 1H)

### Example 10

### 4-{4-[2-(2-Methyl-2H-pyrazol-3-yl)-ethanesulfonylmethyl]-phenoxymethyl}-2-[2-(4-trifluoromethyl-phenyl)-vinyl]-oxazole

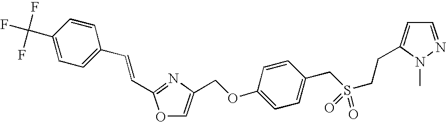

To a solution of 32 mg (0.06 mmol) 4-{4-[2-(2-Methyl-2H-pyrazol-3-yl)-ethanesulfinylmethyl]-phenoxymethyl}-2-[2-(E)-(4-trifluoromethyl-phenyl)-vinyl]-oxazole in 15 ml dichloromethane at 0° C. were added 13 mg (0.06 mmol) 3-chloro-benzenecarboperoxoic acid. The mixture was allowed to warm up and stirred at room temperature overnight. After removal of the solvent purification by HPLC/MS (RP 18, methanol-water-gradient) yielded 30 mg (91%) 4-{4-[2-(2-Methyl-2H-pyrazol-3-yl)-ethanesulfonylmethyl]-phenoxymethyl}-2-[2-(4-trifluoromethyl-phenyl)-vinyl]-oxazole as white solid melting at 204° C.

MS: M=532.3 (ES+)

¹H-NMR (400 MHz, D₆-DMSO): δ=3.04 (t, 8.0 Hz, 2H), 3.35 (t, 8.0 Hz, 2H), 3.74 (s, 3H), 4.46 (s, 2H), 5.05 (s, 2H), 6.11 (d, 1.8 Hz, 1H), 7.09 (d, 8.9 Hz, 2H), 7.31 (d, 16.4 Hz, 1H), 7.34 (d, 8.9 Hz, 2H), 7.36 (d, 1.8 Hz, 1H), 7.62 (d, 16.4 Hz, 1H), 7.76 (d, 8.1 Hz, 2H), 7.95 (d, 8.1 Hz, 2H), 8.27 (s, 1H)

1. A compound according to formula I, 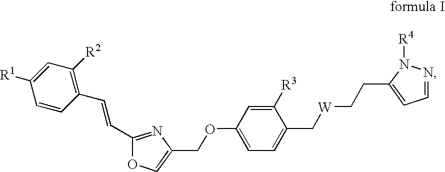
wherein
R¹ is selected from the group consisting of halogenated alkyl, halogenated alkoxy, and halogen;
R² is selected from the group consisting of hydrogen and halogen;
R³ is selected from the group consisting of hydrogen and alkyl;
R⁴ is alkyl; and
W is selected from the group consisting of —O—, —S—, —S(O)—, and —S(O)₂—;
or a pharmaceutically acceptable salt thereof.

2. A compound according to claim 1, wherein
R¹ is halogenated alkoxy; and
R² is hydrogen.

3. A compound according to claim 1, wherein
W is selected from the group consisting of —O— and —S(O)—.

4. A compound according to claim 1, wherein
R³ is alkyl.

5. A compound according to claim 1, selected from the group consisting of:
4-{3-Methyl-4-[2-(2-methyl-2H-pyrazol-3-yl)-ethoxymethyl]-phenoxymethyl}-2-[(E)-2-(4-trifluoromethoxy-phenyl)-vinyl]-oxazole;
4-{3-Methyl-4-[2-(2-methyl-2H-pyrazol-3-yl)-ethoxymethyl]-phenoxymethyl}-2-[(E)-2-(4-trifluoromethyl-phenyl)-vinyl]-oxazole;
2-[(E)-2-(4-Chloro-phenyl)-vinyl]-4-{3-methyl-4-[2-(2-methyl-2H-pyrazol-3-yl)-ethoxymethyl]-phenoxymethyl}-oxazole;
2-[(E)-2-(2-Fluoro-4-trifluoromethyl-phenyl)-vinyl]-4-{3-methyl-4-[2-(2-methyl-2H-pyrazol-3-yl)-ethoxymethyl]-phenoxymethyl}-oxazole;
4-{3-Methyl-4-[2-(2-methyl-2H-pyrazol-3-yl)-ethanesulfinylmethyl]-phenoxymethyl}-2-[(E)-2-(4-trifluoromethoxy-phenyl)-vinyl]-oxazole;
2-[(E)-2-(4-Chloro-phenyl)-vinyl]-4-{3-methyl-4-[2-(2-methyl-2H-pyrazol-3-yl)-ethanesulfinylmethyl]-phenoxymethyl}-oxazole;
4-{3-Methyl-4-[2-(2-methyl-2H-pyrazol-3-yl)-ethanesulfinylmethyl]-phenoxymethyl}-2-[(E)-2-(4-trifluoromethyl-phenyl)-vinyl]-oxazole;
4-{4-[2-(2-Methyl-2H-pyrazol-3-yl)-ethanesulfinylmethyl]-phenoxymethyl}-2-[(E)-2-(4-trifluoromethoxy-phenyl)-vinyl]-oxazole;
4-{4-[2-(2-Methyl-2H-pyrazol-3-yl)-ethanesulfinylmethyl]-phenoxymethyl}-2-[(E)-2-(4-trifluoromethyl-phenyl)-vinyl]-oxazole; and
4-{4-[2-(2-Methyl-2H-pyrazol-3-yl)-ethanesulfonylmethyl]-phenoxymethyl}-2-[(E)-2-(4-trifluoromethyl-phenyl)-vinyl]-oxazole.

6. A process for the manufacture of a compound of formula I, 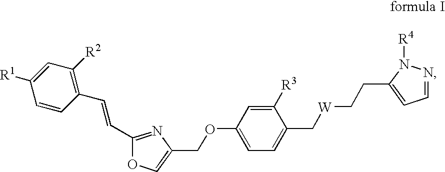
wherein
the compound of formula V, 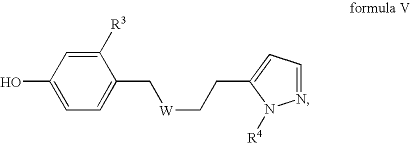
is reacted with a compound of formula IV, 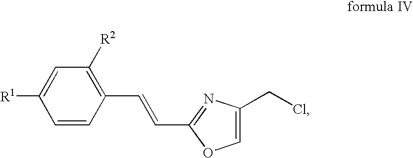
and wherein
R¹ is selected from the group consisting of halogenated alkyl halogenated alkoxy, and halogen;
R² is selected from the group consisting of hydrogen and halogen;
R³ is selected from the group consisting of hydrogen and alkyl;
R⁴ is alkyl; and
W is selected from the group consisting of —O—, —S—, and —S(O)—.

7. A pharmaceutical composition comprising a compound according to claim 1 and a according to claims 1 to 6 together pharmaceutically acceptable carrier.

8-9. (canceled)
